(19) United States
(12) Patent Application Publication
(10) Pub. No.: US 2025/0197947 A1
(43) Pub. Date: Jun. 19, 2025

(54) METHOD AND GENE DETECTION PANEL FOR EVALUATING TREATMENT RESPONSE, RECURRENCE AND SURVIVAL BY DETECTING GENETIC VARIANTS AND THEIR CHANGES BEFORE AND AFTER CONCURRENT CHEMORADIOTHERAPY IN TUMOR TISSUES OF PATIENTS WITH ESOPHAGEAL CANCER

(71) Applicant: LIHPAO LIFE SCIENCE CORP., New Taipei City (TW)

(72) Inventors: Hung-Tai Wu, New Taipei City (TW); Yen-Su Huang, New Taipei City (TW); Hang Chang, New Taipei City (TW); Yu-Jen Chen, New Taipei City (TW); Pei-Wen Yang, New Taipei City (TW)

(21) Appl. No.: 18/978,014

(22) Filed: Dec. 12, 2024

(30) Foreign Application Priority Data
Dec. 13, 2023 (TW) 112148617

(51) Int. Cl. C12Q 1/6886

(52) U.S. Cl. CPC C12Q 1/6886; C12Q 2600/106; C12Q 2600/118; C12Q 2600/156; G01N 2800/52

(57) ABSTRACT

The present disclosure provides a method and a gene detection panel for evaluating treatment response, recurrence and survival by detecting genetic variants and their changes before and after concurrent chemoradiotherapy in tumor tissues of patients with esophageal cancer. The present disclosure develops a set of esophageal cancer NGS analysis panel. Aiming at 402 mutation sites including 35 genes that frequently occur in esophageal squamous cell carcinoma tissue cells, 62 pairs of esophageal squamous cell carcinoma tissues before and after CCRT are analyzed for specific site variation, hoping to find new predictive markers. The present disclosure combines these potential markers into an esophageal cancer detection panel, which has extremely high value for improving the prognosis of esophageal cancer.

### CROSS-REFERENCE TO RELATED APPLICATION

This application claims priority of Taiwan patent application No. 112148617, filed on Dec. 13, 2023, the content of which is incorporated herein in its entirety by reference.

### BACKGROUND OF THE INVENTION

### 1. Field of the Invention

The present invention relates to a method and a gene detection panel for evaluating treatment response, recurrence and survival by detecting genetic variants and their changes before and after concurrent chemoradiotherapy in tumor tissues of patients with esophageal cancer.

### 2. The Prior Art

Esophageal cancer is a deadly disease. Even patients detected early are still at high risk of recurrence at the original site and distant metastasis. Esophageal cancer is mainly classified into esophageal squamous cell carcinoma (ESCC) and adenocarcinoma. The standard treatment for locally advanced esophageal cancer is neoadjuvant concurrent chemoradiotherapy (CCRT) before surgery, with or without surgical resection. Patients with esophageal squamous cell carcinoma who respond well to CCRT have better survival. Under other treatments, the pathological complete response rate is only 10% to 40%. Despite this, the prognosis of esophageal cancer is still poor. Even with the use of combination therapy, the 5-year survival rate is still less than 20%, and more than 50% of patients would develop recurrence at the original site or distant metastasis within two to three years. Tumor, node and metastasis stage (TNM stage) is considered the gold standard for predicting clinical outcomes and guiding treatment strategies. Therefore, multiple reliable simultaneous chemoradiotherapy response and prognostic markers are identified and used to provide more complete treatment information for esophageal cancer patients, which is the focus of researchers in the art.

As mentioned above, esophageal cancer is a deadly disease with a high risk of recurrence, especially if recurrence occurs in patients with esophageal squamous cell carcinoma, the median survival is generally only 8 months. Therefore, developing a more effective gene detection kit for tumor tissue evaluation of preoperative concurrent chemoradiotherapy (CCRT) response and prognosis in patients with esophageal cancer, and understanding the genetic variation before and after treatment would be of great help in improving the treatment effect of esophageal cancer.

### SUMMARY OF THE INVENTION

A primary objective of the present invention is to provide a method for evaluating treatment response, recurrence and survival by detecting genetic variants and their changes before and after concurrent chemoradiotherapy (CCRT) in tumor tissues of a patient with esophageal cancer, comprising the following steps: (a) obtaining a tumor tissue from the patient with esophageal cancer, and extracting genomic DNA from the tumor tissue to obtain at least one genomic DNA sample; and (b) establishing an amplicon library from the at least one genomic DNA sample and performing next generation sequencing (NGS) to obtain at least one genomic DNA datum, thereby evaluating preoperative CCRT response and prognosis in the patient with esophageal cancer; wherein the at least one genomic DNA datum comprises a gene locus related to preoperative CCRT response and a gene locus related to prognosis in the patient with esophageal cancer, wherein when the patient with esophageal cancer having the gene locus related to preoperative CCRT response or the gene locus related to prognosis in the patient with esophageal cancer does achieve 5-year progression-free survival (PFS), indicating poor preoperative CCRT response or poor prognosis; wherein the gene locus related to prognosis in the patient with esophageal cancer comprises Mucin 17 (MUC17) gene locus, p.Asp2397His of Mucin 4 (MUC4) gene, p.His2381Asp of MUC4 gene, p.Pro3360His of MUC4 gene, p.Thr2382Ala of MUC4 gene, p.Thr2411Ser of MUC4 gene, p.Thr3355Ser of MUC4 gene, p.Val3353Ala of MUC4 gene, USH2A gene locus, USH2A loc102723833 p.Leu1658Pro of USH2A gene and myosin, heavy chain 4 (MYH4) gene locus, p.Glu1209Lys of MYH4 gene; wherein the MUC17 gene locus is selected from the group consisting of: p.Thr2702Val of MUC17 gene, p.Thr3355Ser of MUC17 gene, p.Leu2712Val of MUC17 gene, p.Asn2706Ser of MUC17 gene, p.Leu2703_Leu2704delinsProVal of MUC17 gene, p.Pro2716Ala of MUC17 gene, and a combination thereof.

According to an embodiment of the present invention, the gene locus related to preoperative CCRT response is selected from the group consisting of: p.Pro1319Ser of MUC17 gene, p.Arg2159Gly of MUC17 gene, p.Gly1307Ser of MUC17 gene, p.Val1309Met of MUC17 gene, and a combination thereof.

According to an embodiment of the present invention, the esophageal cancer is esophageal squamous cell carcinoma (ESCC).

According to an embodiment of the present invention, the prognosis comprises recurrence and death.

According to an embodiment of the present invention, variation of the p.Pro1319Ser of MUC17 gene in pre-treatment tissue shows that risk of partial response to preoperative CCRT in the patient with esophageal cancer is 6.22-fold than that without variation.

According to an embodiment of the present invention, variation of the p.Thr2702Val of MUC17 gene in pre-treatment tissue shows a 3.32-fold risk of recurrence compared with that without variation.

According to an embodiment of the present invention, variation of the p.Thr2702Val of MUC17 gene in post-treatment tissue shows a 3.21-fold risk of recurrence compared with that without variation.

According to an embodiment of the present invention, compared with the patient with esophageal cancer after CCRT and before CCRT, situation of genetic variation comprises increase and decrease, wherein the increase refers to tissue variation after treatment and no variation before treatment, wherein the decrease refers to tissue variation before treatment and no variation after treatment.

According to an embodiment of the present invention, variation of the combination of p.Pro1319Ser of MUC17 gene and p.Arg2159Gly of MUC17 gene in pre-treatment tissue shows that risk of partial response to preoperative CCRT in the patient with esophageal cancer is 7-fold than that without variation.

According to an embodiment of the present invention, variation of the combination of p.Pro1319Ser of MUC17 gene and p.Gly1307Ser of MUC17 gene in pre-treatment tissue shows that risk of partial response to preoperative CCRT in the patient with esophageal cancer is 6.03-fold than that without variation.

According to an embodiment of the present invention, variation of the combination of p.Pro1319Ser of MUC17 gene and p.Val1309Met of MUC17 gene in pre-treatment tissue shows that risk of partial response to preoperative CCRT in the patient with esophageal cancer is 6.35-fold than that without variation.

Another objective of the present invention is to provide a gene detection panel for evaluating treatment response, recurrence and survival by detecting genetic variants and their changes before and after concurrent chemoradiotherapy (CCRT) in tumor tissues of a patient with esophageal cancer, which is established by the aforementioned method.

Another objective of the present invention is to provide a method for tumor tissue evaluation of changes in gene locus variation, correlation with treatment response and prognosis in a patient with esophageal cancer before and after concurrent chemoradiotherapy (CCRT), comprising the following steps: (a) obtaining a tumor tissue from the patient with esophageal cancer before and after CCRT treatment, and extracting genomic DNA from the tumor tissue to obtain at least one genomic DNA sample; and (b) establishing an amplicon library from the at least one genomic DNA sample and performing next generation sequencing (NGS) to obtain at least one genomic DNA datum, thereby evaluating changes in gene locus variation in the patient with esophageal cancer before and after CCRT; wherein the at least one genomic DNA datum comprises a gene locus related to CCRT response, wherein the changes in gene locus variation in the patient with esophageal cancer having the gene locus related to CCRT response before and after CCRT response are statistically significant; wherein the gene locus related to CCRT response associated with treatment response is selected from the group consisting of: p.Glu1523Lys of EP300 gene, p.Glu5905Asp of SYNE1 gene, p.Asp2397His of MUC4 gene, p.Ala2409Val of MUC4 gene, p.Glu5905Asp of SYNE1 gene, p.Ala2409Val of MUC4 gene, and a combination thereof; wherein the gene locus related to CCRT response associated with recurrence is selected from the group consisting of: p.Glu5905Asp of SYNE1 gene, p.Pro3360His of MUC4 gene, p.Thr2382Ala of MUC4 gene, p.Ala2390Thr of MUC4 gene, p.Leu2712Val of MUC17 gene, and a combination thereof; wherein the gene locus related to CCRT response associated with survival is selected from the group consisting of: p.Asp2397His of MUC4 gene, p.Pro3360His of MUC4 gene, p.Ala2390Thr of MUC4 gene, and a combination thereof.

According to an embodiment of the present invention, compared with the patient with esophageal cancer after CCRT and before CCRT, situation of genetic variation comprises increase and decrease, wherein the increase refers to tissue variation after treatment and no variation before treatment, wherein the decrease refers to tissue variation before treatment and no variation after treatment.

In summary, the present invention develops a next generation sequencing (NGS) kit for esophageal cancer (especially esophageal squamous cell carcinoma). Therefore, we first analyzed 402 genetic variants including 35 genes that commonly occur in esophageal squamous cell carcinoma tissue cells, and analyzed the site-specific variants in 62 pairs of esophageal squamous cell carcinoma tissues before and after CCRT to find new predictive markers. It was found that the variations at some sites would change before and after CCRT treatment, and the pattern of changes is significantly related to treatment response, recurrence and survival. The present invention combines these potential markers into an esophageal cancer detection kit, which is of extremely high value for improving the prognosis of esophageal cancer.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows that receiver operating characteristic (ROC) curve for indicated combining risk variations was used to differentiate the esophageal squamous cell carcinoma (ESCC) patients with complete or partial response to CCRT. AUC, area under the ROC curve.

FIGS. 2A and 2B show that receiver operating characteristic (ROC) curves for risk genotypes of the tissues before CCRT were used to differentiate the ESCC patients with (2A) disease recurrence or non-recurrence and (2B) dead or alive. AUC, area under the ROC curve.

FIGS. 3A and 3B show Kaplan-Meier estimates of tumor progression-free survival (PFS, A) and overall survival (OS, B) by patients carrying unfavorable genotypes (positive) or not (negative) in tissue before CCRT. Unfavorable genotypes: MUC17 p.Thr2702Val(variant) and MUC4 p.Thr3355Ser(wild-type); MST, median survival time.

FIG. 4 shows an overview of tissue-site variation changes before and after CCRT, in which there are 1814 total variation sites before CCRT, and 1754 total variation sites after CCRT. The difference in variation amount between the post-CCRT and the pre-CCRT is −60.

FIG. 5 shows the order of the number of increases in site variation after CCRT (≥1), that is, the total increase in site variation after CCRT is greater than 1 sample pair. The genes with the top 10 most frequently increased variants are USH2A, MUC4 (2), MUC17 (3), EP300, LRP2, SYNE1 and TP53.

FIG. 6 shows the order of the number of sites with reduced variation after CCRT (≥1), that is, the reduction in site variation after CCRT is greater than 1 sample pair. The genes with the top 10 most frequently increased variants are MUC4 (4), MUC17 (4), ZFHX4 and TP53.

FIG. 7 shows that reduced MUC17p.Leu2712Val variation after CCRT is significantly associated with risk of shorter PFS. Variation decrease refers to tissue variation before treatment and no variation in tissue after treatment.

FIGS. 8-11 show that there are four MUC17 loci after CCRT, including MUC17p.Leu2712Val, MUC17p.Leu2703_Leu2704delinsProVa, MUC17p.Asn2706Ser and MUC17p.Pro2716Ala. The unchanged variation amount of the four MUC17 loci is significantly related to better overall survival (OS) time.

FIG. 12 shows that the reduced variation at the MUC4p.Pro3360His locus after CCRT is significantly associated with longer overall survival (OS) time. Variation decrease refers to tissue variation before treatment and no variation in tissue after treatment.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

### Example 1

### Patients Characteristics

Study population in this example is described as follows. A total number of 62 patients with locally advanced esophageal squamous cell carcinoma (ESCC) (T3N0-1M0 or T1-3N1M0) and received neoadjuvant (preoperative) concurrent chemoradiotherapy (CCRT) followed by esophagectomy were enrolled and retrospectively reviewed from MacKay Memorial Hospital in Taiwan with approved by the hospital's ethical committee. The prescribed radiation dose for large tumor and metastatic lymph nodes or subclinical mucosal/submucosal disease and regional lymphatic basin were 48 Gy and 43.2 Gy, respectively. All patients underwent the intensity modulated radiation therapy in 24 fractions and received concurrent chemotherapy with cisplatin.

Among the patients, 22 (35.5%) had complete response to neoadjuvant CCRT (defined as tumor regression grade (pathological complete response, pCR), and the rest of enrolled patients (N=40, 64.5%) only had partial response to the treatment (PR). Information regarding the demographic and clinical data were obtained from medical record and the clinical database in the Dept. of Surgery and Department of Pathology and Laboratory Medicine in MacKay Memorial Hospital.

The formalin-fixed paraffin-embedded (FFPE) pathologic esophageal tumor tissue specimens before treatment of CCRT were collected from endoscopic biopsies. The post CCRT FFPE tissues used for both pathological response evaluation and genomic analysis were collected during esophagectomy.

The patients' characteristics are shown in Table 1. Of 62 patients, 58 (93.5%) were male, 38 (61.3%) with T3 clinical tumor stage (cT), 32 patients had N0 or N1 clinical lymphonode (cN) stage, and the resulting clinical stage (cStage) of stages 2 and 3 was 29% and 71.0% respectively. There was no significant difference in the distribution of these clinical or demographic variables between the groups of partial response (PR) or pathological complete response (pCR) (Table 1).

TABLE 1

| | | | | PR | | pCR | | p- |
|---|---|---|---|---|---|---|---|---|
| | variable | N | | 40 (64.5) | | N = 22 (35.5) | | value* |
| Sex | | | | | | | | 0.124 |
| | Male | 58 | (93.5) | 39 | (67.2) | 19 | (32.8) | |
| | Female | 4 | (6.5) | 1 | (25.0) | 3 | (75.0) | |
| cT | | | | | | | | 0.190 |
| | T1 + T2 | 18 | (29.0) | 11 | (61.1) | 7 | (38.9) | |
| | T3 | 38 | (61.3) | 23 | (60.5) | 15 | (39.5) | |
| | T4 | 6 | (9.7) | 6 | (100.0) | 0 | (0) | |
| cN | | | | | | | | 0.142 |
| | N0 + | 32 | (51.6) | 22 | (68.8) | 10 | (31.3) | |
| | N1 | | | | | | | |
| | N2 | 22 | (35.5) | 11 | (50.0) | 11 | (50.0) | |
| | N3 | 8 | (12.9) | 7 | (87.5) | 1 | (12.5) | |
| cStage | | | | | | | | 0.561 |
| | cStage | 18 | (29.0) | 13 | (72.2) | 5 | (27.8) | |
| | II | | | | | | | |
| | cStage | 44 | (71.0) | 27 | (61.4) | 17 | (38.6) | |
| | III | | | | | | | |

*Pearson's Chi-square test or Fisher's exact test

### Example 2

### CCRT Response-Relevant Genetic Variants

The procedure regarding analysis of genetic variants by next generation sequencing (NGS) in this example is as follows. Analysis of genetic variations of the ESCC tissue by NGS were performed in the laboratories of LIHPAO Life Science Co. The workflow included the steps of genomic DNA extraction, library preparation, sequencing and data analysis. Each step was described as previous report (Tang P, Tan C, Pang Q, Chi C W, Wang Y, Yuan Z, Huang Y C, Chen Y J. Combination of 35-Gene Mutation Profile and Radiotherapy Dosimetry Predicts the Therapeutic Outcome of Definitive Chemoradiation in Patients With Esophageal Squamous Cell Carcinoma. Front Oncol. 2021 Aug. 27; 11:729418) and briefly described as follows.

The procedure regarding genomic DNA extraction in this example is as follows. For each specimen, 5×5 mm² with 5-μm thick FFPE (Formalin-Fixed Paraffin-Embedded) sections were cut from block. Genomic DNA was extracted using Cobas® DNA Sample Preparation Kit (Roche, Basel, Switzerland) according to the manufacturer's instructions. Isolated DNA was quantified using the Qubit dsDNA HS Assay kit (Thermo Fisher Scientific, Waltham, MA, USA). DNA concentration and integrity were analyzed by using DNF-474 High Sensitivity NGS Fragment Analysis Kit (AATI, Ankeny, IA, USA) and Fragment Analyzer Automated CE System.

The procedure regarding amplicon library construction and NGS is as follows. DNA with the amount of 10 ng from FFPE tissue sample was used to construct amplicon library using Ion AmpliSeq™ Library Kit 2.0 (Thermo Fisher Scientific), and Ion Xpress™ Barcode Adapters Kit (Thermo Fisher Scientific). Library purification was carried out by Agencourt AMPure XP reagent (Beckman Coulter, Brea, CA, USA) and washed with 70% ethanol on DynaMag™-2 Magnet (Thermo Fisher Scientific). Quality control of the libraries was established using Ion Library TaqMan Quantitation Kit collocation with 7500 Fast along with 7500 Real-Time PCR System (Thermo Fisher Scientific). The ESCC panel comprising 402 genetic variants, 159 amplicons, which covering 35 genes including ABCA13, DNAH5, FBXW7, FAT1, FAT3, GPR98, EP300, DMD, KDM6A, CSMD3, CDKN2A, KMT2D, MUC4, MUC17, MUC2, MUC16, MYH4, TNN, HMCN1, USH2A, LRP1B, XIRP2, LRP2, NFE2L2, NOTCH1, TTN, FSIP2, SI, PIK3CA, RB1, TP53, ZFHX4, TRIO, SYNE1, and PCLO. The quantified libraries were clonally amplified on ion sphere particles by emulsion polymerase chain reaction using the Ion OneTouch™ 2 system with the Ion PGM Hi-Q View OT2 Kit (Thermo Fisher Scientific). Next, the ion sphere particles were enriched in an Ion OneTouch™ ES instrument (Thermo Fisher Scientific). Finally, the enriched ion sphere particles were loaded onto the 316 chip, and sequencing was performed on an Ion Torrent PGM system (Ion Torrent, Paisley, UK).

The personal genome machine-based DNA sequencing data were generated using Torrent Suite software (Thermo Fisher Scientific). Variant calling and annotation were conducted using Ion-Reporter v5.1.0. Variations with an average coverage of ≥1500 reads and a variant allele frequency of ≥5% were reported.

The statistical analysis in this example is described as follows. The distribution of demographic, clinical characteristics, and variant genotypes among the subgroups with different clinical results, including treatment response, recurrence and mortality were analyzed by Pearson's Chi-square test or Fisher's exact test if anyone of each cell counts in the cross table is less than 5. Univariate or multivariate logistic regression was used to evaluate the odds ratios (ORs) of partially response (PR) to CCRT. The hazard ratios (HRs) obtained from Cox regression analysis was used to described the relative risk of recurrence or death. Data were expressed as mean value and 95% confidence interval (CI) for regression analysis. Correlations between variant genotypes and both overall survival (OS) and progression-free survival (PFS) were also be analyzed using the Kaplan-Meier survival function and compared using the log-rank test. The liner relationship between genotypic variable was analyzed by collinearity diagnostics using variance inflation factor (VIF). All statistical analyses was conducted with IBM SPSS Statistics for Windows, version 25.0 (IBM Corp., Armonk, N.Y., USA). A p-value ≤0.05 was considered statistically significant.

Table 2 demonstrates there were 19 variants detected from tumor tissue before CCRT exhibited significant (P≤0.05) to be associated with CCRT response by Pearson's Chi-square test or Fisher's exact test. Among these genetic variants, 14 were within the coding region of the MUC17 gene. Univariant logistic regression further revealed 11 variants, including FAT1 p.Asn2678Asp, MUC17 p.Ala1322Thr, MUC17 p.Arg2159Gly, MUC17 p.Asn2706Ser, MUC17 p.Gly1307Ser, MUC17 p.Leu2712Val, MUC17 p.Pro1319Ser, MUC17 p.Pro1321Thr, MUC17 p.Thr2702Val, MUC17 p.Thr2721Ile, and MUC17 p.Val1309Met, significantly correlated with the increased risk of partial response.

TABLE 2

| | Varia- | | | | | |
|---|---|---|---|---|---|---|
| | tion | | | | | |
| | (0: No; | PR | | pCR | | p- |
| Variants | 1: Yes) | 40(64.5) | | 22 (35.5) | | value* |
| FAT p.Asn2678Asp | 0 | 4 | (33.3) | 8 | (66.7) | 0.019 |
| | 1 | 36 | (72.0) | 14 | (28.0) | |
| MUC17 p.Ala1322Thr | 0 | 4 | (36.4) | 7 | (63.6) | 0.042 |
| | 1 | 36 | (70.6) | 15 | (29.4) | |
| MUC17 p.Arg2159Gly | 0 | 19 | (51.4) | 18 | (48.6) | 0.014 |
| | 1 | 21 | (84.0) | 4 | (16.0) | |
| MUC17 p.Asn2706Ser | 0 | 26 | (56.5) | 20 | (43.5) | 0.034 |
| | 1 | 14 | (87.5) | 2 | (12.5) | |
| MUC17 p.Gly1307Ser | 0 | 20 | (54.1) | 17 | (45.9) | 0.057 |
| | 1 | 20 | (80.0) | 5 | (20.0) | |
| MUC17 p.Leu2703_leu | 0 | 27 | (57.4) | 20 | (42.6) | 0.062 |
| | 1 | 13 | (86.7) | 2 | (13.3) | |
| MUC17 p.Leu2712Val | 0 | 26 | (56.5) | 20 | (43.5) | 0.034 |
| | 1 | 14 | (87.5) | 2 | (12.5) | |
| MUC17 p.Pro1319Ser | 0 | 12 | (42.9) | 16 | (57.1) | 0.001 |
| | 1 | 28 | (82.4) | 6 | (17.6) | |
| MUC17 p.Pro1321Thr | 0 | 14 | (46.7) | 16 | (53.3) | 0.004 |
| | 1 | 26 | (81.3) | 6 | (18.8) | |
| MUC17 p.Pro2716Ala | 0 | 27 | (57.4) | 20 | (42.6) | 0.062 |
| | 1 | 13 | (86.7) | 2 | (13.3) | |
| MUC17 p.Ser2785Ala | 0 | 24 | (57.1) | 18 | (42.9) | 0.095 |
| | 1 | 16 | (80.0) | 4 | (20.0) | |
| MUC17 p.Thr2702Val | 0 | 19 | (52.8) | 17 | (47.2) | 0.032 |
| | 1 | 21 | (80.8) | 5 | (19.2) | |
| MUC17 p.Thr2721Ile | 0 | 12 | (48.0) | 13 | (52.0) | 0.025 |
| | 1 | 28 | (75.7) | 9 | (24.3) | |
| MUC17 p.Thr2802Ile | 0 | 33 | (60.0) | 22 | (40.0) | 0.044 |
| | 1 | 7 | (100.0) | 0 | (0) | |
| MUC17 p.Val1309Met | 0 | 19 | (52.8) | 17 | (47.2) | 0.032 |
| | 1 | 21 | (80.8) | 5 | (19.2) | |
| MUC4 p.Ala2409Val | 0 | 39 | (68.4) | 18 | (31.6) | 0.049 |
| | 1 | 1 | (20.0) | 4 | (80.0) | |
| MYH4 p.Gln1210fs | 0 | 33 | (70.2) | 14 | (29.8) | 0.097 |
| | 1 | 7 | (46.7) | 8 | (53.3) | |
| PIK3CA p.Gly1049Cys | 0 | 13 | (52.0) | 12 | (48.0) | 0.090 |
| | 1 | 27 | (73.0) | 10 | (27.0) | |
| TTN p.Asp7145His | 0 | 11 | (50.0) | 11 | (50.0) | 0.076 |
| | 1 | 29 | (72.5) | 11 | (27.5) | |

*Pearson's Chi-square test or Fisher's exact test

Of these variants, MUC17 p.Pro1319Ser exhibited the most predominant correlation with a 6.22-fold increased risk for partial response (OR [95% CI]=6.22 [1.96-19.78], p=0.002, Table 3).

TABLE 3

| | | |
|---|---|---|
| Variants | OR (95% CI)# | P-value\hat{ } |

| | | | |
|---|---|---|---|
| FAT1 p.Asn2678Asp | 5.14 | (1.33-19.83) | 0.017* |
| MUC17 p.Ala1322Thr | 4.2 | (1.07-16.50) | 0.040* |
| MUC17 p.Arg2159Gly | 4.97 | (1.42-17.34) | 0.012* |
| MUC17 p.Asn2706Ser | 5.39 | (1.10-26.46) | 0.038* |
| MUC17 p.Gly1307Ser | 3.40 | (1.05-11.00) | 0.041* |
| MUC17 p.Leu2703_leu | 4.82 | (0.98-23.78) | 0.054 |
| MUC17 p.Leu2712Val | 5.39 | (1.10-26.46) | 0.038* |
| MUC17 p.Pro1319Ser | 6.22 | (1.96-19.78) | 0.002** |
| MUC17 p.Pro1321Thr | 4.95 | (1.58-15.50) | 0.006** |
| MUC17 p.Pro2716Ala | 4.82 | (0.98-23.78) | 0.054 |
| MUC17 p.Ser2785Ala | 3.00 | (0.86-10.52) | 0.086 |
| MUC17 p.Thr2702Val | 3.76 | (1.16, 12.16) | 0.027* |
| MUC17 p.Thr2721Ile | 3.37 | (1.13-9.99) | 0.028* |

| | | |
|---|---|---|
| MUC17 p.Thr2802Ile | — | 0.999 |

| | | | |
|---|---|---|---|
| MUC17 p.Val1309Met | 3.76 | (1.16-12.16) | 0.027* |
| MUC4 p.Ala2409Val | 0.12 | (0.01-1.11) | 0.061 |
| MYH4 p.Gln1210fs | 0.37 | (0.11-1.22) | 0.103 |
| PIK3CA p.Gly1049Cys | 2.49 | (0.86-7.26) | 0.094 |
| TTN p.Asp7145His | 2.64 | (0.89-7.81) | 0.080 |

#Hazard ratio (HR [95% confidence interval (CI)]of tumor recurrence
\hat{ }Univariate Cox regression
*P < 0.05;
**p < 0.01

We analyzed the collinearity of unfavorable variants for CCRT response within the MUC17 gene with collinearity diagnostics by examining the variance inflation factor (VIF). Three variants with a predominant unfavorable effect, including MUC17 p.Arg2159Gly, p.Gly1307Ser, and p.Val1309Met, exhibited a high degree of genetic collinearity (Table 4).

TABLE 4

| Variants | VIF |
|---|---|
| MUC17 p.Arg2159Gly | 15.852 |
| MUC17 p.Gly1307Ser | 8.131 |
| MUC17 p.Val1309Met | 24.117 |

VIF, Variance Inflation Factor

Combining each of these 3 variants with p.Pro1319, patients carrying at least one of the variations (positive) showed obviously increased partial response group compared to those carrying none of the variation (negative), especially the combination of MUC17 p.Pro1319Ser and p.Arg2159Gly (PR vs. pCR, 80% vs. 31.8%, p<0.001, Table 5).

TABLE 5

| | Unfavorable | PR | | pCR | | |
|---|---|---|---|---|---|---|
| Variants | genotypes | 40 (64.5) | | 22 (35.5) | | p-value* |
| MUC17 p.Pro1319Ser + | Negative | 8 | (20.0) | 15 | (68.2) | <0.001 |
| p.Arg2159Gly | Positive | 32 | (80.0) | 7 | (31.8) | |
| MUC17 p.Pro1319Ser + | Negative | 9 | (22.5) | 14 | (63.6) | 0.002 |
| p.Gly1307Ser | Positive | 31 | (77.5) | 8 | (36.4) | |
| MUC17 p.Pro1319Ser + | Negative | 8 | (20.0) | 14 | (63.6) | 0.001 |
| p.Val1309Met | Positive | 32 | (80.0) | 8 | (36.4) | |

*Pearson's Chi-square test or Fisher's exact test

Univariate logistics regression also demonstrated the pronounced effect of MUC17 p.Pro1319Ser and p.Arg2159Gly with a 7-fold increased risk for partial response (OR [95% CI]=7.00 [3.07-15.94], p<0.001, Table 6).

TABLE 6

| | Unfavorable | | |
|---|---|---|---|
| Variants | genotypes | OR (95% CI) | p-value* |
| MUC17 p.Pro1319Ser + | Negative | 1 | |
| p.Arg2159Gly | Positive | 7.00 (3.07-15.94) | <0.001 |
| MUC17 p.Pro1319Ser + | Negative | 1 | |
| p.Gly1307Ser | Positive | 6.03 (2.69-13.52) | <0.001 |
| MUC17 p.Pro1319Ser + | Negative | 1 | |
| p. Val1309Met | Positive | 6.35 (2.81-14.39) | <0.001 |

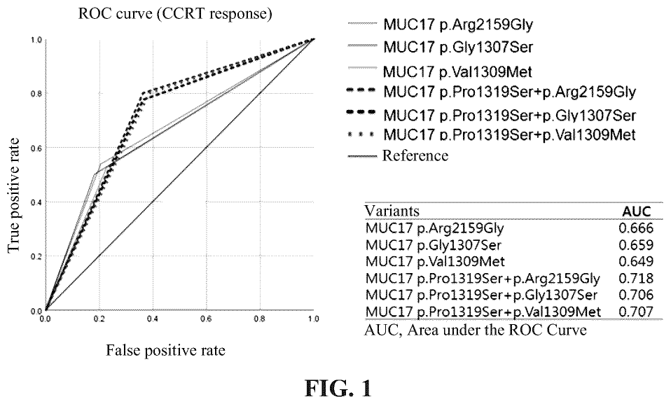

FIG. 1 shows that receiver operating characteristic (ROC) curve for indicated combining risk variations was used to differentiate the esophageal squamous cell carcinoma (ESCC) patients with complete or partial response to CCRT. AUC, area under the ROC curve.

ROC curve revealed that the positive of either MUC17 p.Pro1319Ser or p.Arg2159Gly had a fair capability for predicting CCRT response (AUC=0.718, FIG. 1).

### Example 3

### Prognostic Relevant Genetic Variants

We further analyzed the correlation between genetic variation and prognosis, including tumor recurrence and mortality. Table 7 demonstrates that there were 24 and 16 variants detected in tumor tissue before CCRT showed a significant correlation with tumor recurrence. Most variants were within the coding regions of the MUC17 and MUC4 genes.

TABLE 7

| | Variation | | | | | | | | | | | |
|---|---|---|---|---|---|---|---|---|---|---|---|---|
| | (0: No; | No recurrence | | Recurrenc | | *p- | Varinats | No recurrence | | Recurrenc | | *p- |
| Varinats (before CCRT) | 1: Yes) | 11 (17.7) | | 51 (82.3) | | value | (after CCRT) | 11 (17.7) | | 51 (82.3) | | value |
| EP300 p.Glu1523Lys | 0 | 6 | (11.5) | 46 | (88.5) | 0.011 | EP300 p.Glu1523Lys | 4 | (8.7) | 42 | (91.3) | 0.004 |
| | 1 | 5 | (50.0) | 5 | (50.0) | | | 7 | (43.8) | 9 | (56.3) | |
| MIR548N\|TTN-AS1\|TTN | 0 | 2 | (66.7) | 1 | (33.3) | 0.079 | | | | | | |
| \|\|p.Thr19762Ile | 1 | 9 | (15.3) | 50 | (84.7) | | | | | | | |
| MIR548N\|TTN-AS1\|TTN | 0 | 2 | (66.7) | 1 | (33.3) | 0.079 | | | | | | |
| \|\|p.Thr21880Ile | 1 | 9 | (15.3) | 50 | (84.7) | | | | | | | |
| MUC16 p.Thr5382Lys | 0 | 7 | (12.7) | 48 | (87.3) | 0.015 | MUC16 p.Thr5382Lys | 7 | (13.2) | 46 | (86.8) | 0.044 |
| | 1 | 4 | (57.1) | 3 | (42.9) | | | 4 | (44.4) | 5 | (55.6) | |
| MUC17 p.Asn2706Ser | 0 | 11 | (23.9) | 35 | (76.1) | 0.052 | | | | | | |
| | 1 | 0 | (0) | 16 | (100.0) | | | | | | | |
| MUC17 p.Gly2906Ala | 0 | 9 | (15.3) | 50 | (84.7) | 0.079 | | | | | | |
| | 1 | 2 | (66.7) | 1 | (33.3) | | | | | | | |
| MUC17 p.Leu2712Val | 0 | 11 | (23.9) | 35 | (76.1) | 0.052 | | | | | | |
| | 1 | 0 | (0) | 16 | (100.0) | | | | | | | |
| MUC17 p.Lys1306Asn | 0 | 8 | (14.0) | 49 | (86.0) | 0.035 | | | | | | |
| | 1 | 3 | (60.0) | 2 | (40.0) | | | | | | | |
| MUC17 p.Thr2702Val | 0 | 11 | (30.6) | 25 | (69.4) | 0.002 | MUC17 p.Thr2702Val | 11 | (28.2) | 28 | (71.8) | 0.003 |
| | 1 | 0 | (0) | 26 | (100.0) | | | 0 | (0) | 23 | (100.0) | |
| MUC17 p.Thr2721Ile | 0 | 8 | (32.0) | 17 | (68.0) | 0.021 | | | | | | |
| | 1 | 3 | (8.1) | 34 | (91.9) | | | | | | | |
| MUC17 p.Asn2706Ser | 0 | 11 | (23.9) | 35 | (76.1) | 0.052 | | | | | | |
| | 1 | 0 | (0) | 16 | (100.0) | | | | | | | |
| MUC17 p.Leu2703_Leu27( | 0 | 11 | (23.4) | 36 | (76.6) | 0.052 | | | | | | |
| | 1 | 0 | (0) | 15 | (100.0) | | | | | | | |
| MUC17 p.Pro2716Ala | 0 | 11 | (23.4) | 36 | (76.6) | 0.052 | | | | | | |
| | 1 | 0 | (0) | 15 | (100.0) | | | | | | | |
| MUC17 p.Thr2721Ile | 0 | 8 | (32.0) | 17 | (68.0) | 0.021 | | | | | | |
| | 1 | 3 | (8.1) | 34 | (91.9) | | | | | | | |
| MUC4 p.Ala2390Thr | 0 | 7 | (12.7) | 48 | (87.3) | 0.015 | MUC4 p.Ala2390Thr | 8 | (13.8) | 50 | (86.2) | 0.016 |
| | | 4 | (57.1) | 3 | (42.9) | | | 3 | (75.0) | 1 | (25.0) | |
| | | | | | | | MUC4 p.Asp2405Asn | 9 | (15.3) | 50 | (84.7) | 0.079 |
| | | | | | | | | 2 | (66.7) | 1 | (33.3) | |
| MUC4 p.Asp2397His | 0 | 6 | (11.1) | 48 | (88.9) | 0.003 | | | | | | |
| | 1 | 5 | (62.5) | 3 | (37.5) | | | | | | | |
| MUC4 p.Gly3372Asp | 0 | 7 | (13.5) | 45 | (86.5) | 0.067 | MUC4 p.Gly3372Asp | 5 | (10.0) | 45 | (90.0) | 0.004 |
| | 1 | 4 | (40.0) | 6 | (60.0) | | | 6 | (50.0) | 6 | (50.0) | |
| MUC4 p. His2381Asp | 0 | 6 | (11.1) | 48 | (88.9) | 0.003 | MUC4 p. His2381 Asp | 8 | (14.3) | 48 | (85.7) | 0.063 |
| | 1 | 5 | (62.5) | 3 | (37.5) | | | 3 | (50.0) | 3 | (50.0) | |
| MUC4 p. His2413Gln | 0 | 8 | (14.0) | 49 | (86.0) | 0.035 | MUC4 p. His2413Gln | 9 | (15.3) | 50 | (84.7) | 0.079 |
| | 1 | 3 | (60.0) | 2 | (40.0) | | | 2 | (66.7) | 1 | (33.3) | |
| MUC4 p. Pro3360His | 0 | 4 | (9.3) | 39 | (90.7) | 0.026 | | | | | | |
| | 1 | 7 | (36.8) | 12 | (63.2) | | | | | | | |
| MUC4 p.Ser3370Thr | 0 | 8 | (14.3) | 48 | (85.7) | 0.063 | MUC4 p.Ser3370Thr | 7 | (12.7) | 48 | (87.3) | 0.015 |
| | 1 | 3 | (50.0) | 3 | (50.0) | | | 4 | (57.1) | 3 | (42.9) | |
| MUC4 p. Thr2382Ala | 0 | 5 | (10.2) | 44 | (89.8) | 0.007 | MUC4 p. Thr2382Ala | 6 | (11.5) | 46 | (88.5) | 0.011 |
| | 1 | 6 | (46.2) | 7 | (53.8) | | | 5 | (50.0) | 5 | (50.0) | |
| MUC4 p. Thr2398Ala | 0 | 8 | (13.6) | 51 | (86.4) | 0.004 | MUC4 p. Thr2398Ala | 8 | (13.8) | 50 | (86.2) | 0.016 |
| | 1 | 3 | (100.0) | 0 | (0) | | | 3 | (75.0) | 1 | (25.0) | |
| MUC4 p. Thr2411Ser | 0 | 2 | (5.1) | 37 | (94.9) | 0.001 | MUC4 p. Thr2411Ser | 3 | (6.8) | 41 | (93.2) | 0.001 |
| | 1 | 9 | (39.1) | 14 | (60.9) | | | 8 | (44.4) | 10 | (55.6) | |
| MUC4 p. Thr3350Asn | 0 | 8 | (14.3) | 48 | (85.7) | 0.063 | MUC4 p. Thr3350Asn | 7 | (12.7) | 48 | (87.3) | 0.015 |
| | 1 | 3 | (50.0) | 3 | (50.0) | | | 4 | (57.1) | 3 | (42.9) | |
| MUC4 p. Thr3355Ser | 0 | 0 | (0) | 31 | (100.0) | <0.001 | MUC4 p. Thr3355Ser | 0 | (0) | 23 | (100.0) | 0.003 |
| | 1 | 11 | (35.5) | 20 | (64.5) | | | 11 | (28.2) | 28 | (71.8) | |
| MUC4 p. Val3353Ala | 0 | 5 | (10.6) | 42 | (89.4) | 0.018 | MUC4 p. Val3353 Ala | 5 | (10.9) | 41 | (89.1) | 0.052 |
| | 1 | 6 | (40.0) | 9 | (60.0) | | | 6 | (37.5) | 10 | (62.5) | |
| | | | | | | | USH2A\|LOC102723833 | 0 | (0) | 17 | (100.0) | 0.026 |
| | | | | | | | p.Leu1658Pro\| | 11 | (24.4) | 34 | (75.6) | |

*Pearson's Chi-square test or Fisher's exact test

Most variants were within the coding region of MUC17 and MUC4 gene. Univariant logistic regression further revealed 15 variants of the tissue before CCRT, including MUC17 p.Asn2706Ser, MUC17 p.Leu2712Val, MUC17 p.Thr2702Val, MUC17 p.Thr2721Ile, MUC17 p.Asn2706Ser, MUC17p.Leu2703_Leu2704 delinsProVa, MUC17 p.Pro2716Ala, MUC17 p.Thr2721Ile, MUC4 p.Asp2397His, MUC4 p.His2381Asp, MUC4 p.Pro3360His, MUC4 p.Thr2382Ala, MUC4 p.Thr2411Ser, MUC4 p.Thr3355Ser, and MUC4 p.Val3353Ala, are significantly correlated with the increased risk for disease recurrence (see Table 8). In the analysis of the tissue after CCRT, 10 variants among the genes, including EP300, MUC17, MUC4 and USH2A, exhibited evidently increased risk for recurrence of patients (Table 8). Among these variants, MUC17 p.Thr2702Val exhibited the most pronounced unfavorable effect on the prognosis from the tissue both before and after CCRT. Compared with those without genetic variants, patients with genetic variants have a 3.32-fold and 3.21-fold increased risk for recurrence respectively (before CCRT, OR[95% CI]=3.32 (1.84-6.00), p<0.001; after CCRT, OR[95% CI]=3.21 (1.80-5.76), p<0.001, Table 8). Meanwhile, the variation of MUC4 p.Thr3355Ser exhibited evidently effect on reduced risk of recurrence (before CCRT, OR[95% CI]=0.39 (0.21-0.69), p=0.002; after CCRT, OR[95% CI]=0.45 (0.26-0.80), p=0.006, Table 8).

TABLE 8

| | HR (95% CI)# | | HR (95% CI)# | |
|---|---|---|---|---|
| Variants | before CCRT | P-value\hat{ } | after CCRT | P-value\hat{ } |
| EP300 p.Glu1523Lys | 0.41 (0.16-1.03) | 0.058 | 0.40 (0.19-0.82) | 0.013 |
| MIR548N\|TTN-AS1\|TTN | 5.09 (0.70-37.10) | 0.108 | 0.28 (0.04-2.14) | 0.221 |
| \|\|p.Thr19762Ile | | | | |
| MIR548N\|TTN-AS1\|TTN | 5.09 (0.70-37.10) | 0.108 | — | — |
| \|\|p.Thr21880Ile | | | | |
| MUC16 p.Thr5382Lys | 0.32 (0.10-1.01) | 0.051 | 0.51 (0.20-1.29) | 0.157 |
| MUC17 p.Asn2706Ser | 3.85 (2.03-7.32) | <0.001 | 1.95 (0.99-3.86) | 0.055 |
| MUC17 p.Gly2906Ala | 0.22 (0.03-1.57) | 0.129 | 1.11 (0.34-3.60) | 0.859 |
| MUC17 p.Leu2712Val | 3.52 (1.86-6.69) | <0.001 | 1.80 (0.89-3.64) | 0.102 |
| MUC17 p.Lys1306Asn | 0.35 (0.09-1.45) | 0.149 | 1.11 (0.34-3.60) | 0.859 |
| MUC17 p.Thr2702Val | 3.32 (1.84-6.00) | <0.001 | 3.21 (1.80-5.76) | <0.001 |
| MUC17 p.Thr2721Ile | 2.28 (1.27-4.11) | 0.006 | 1.86 (1.01-3.41) | 0.045 |
| MUC17 p.Asn2706Ser | 3.85 (2.03-7.32) | <0.001 | 1.95 (0.99-3.86) | 0.055 |
| MUC17p.Leu2703_Leu2704 | 3.61 (1.89-6.90) | <0.001 | 1.80 (0.89-3.64) | 0.102 |
| delinsProVa | | | | |
| MUC17 p.Pro2716Ala | 3.61 (1.89-6.90) | <0.001 | 1.80 (0.89-3.64) | 0.102 |
| MUC17 p.Thr2721Ile | 2.28 (1.27-4.11) | 0.005 | 1.86 (1.01-3.41) | 0.045 |
| MUC4 p.Ala2390Thr | 0.34 (0.11-1.10) | 0.072 | 0.17 (0.02-1.25) | 0.082 |
| MUC4 p.Asp2397His | 0.28 (0.09-0.91) | 0.034 | 0.46 (0.11-1.91) | 0.285 |
| MUC4 p.Asp2405Asn | 0.67 (0.16-2.76) | 0.577 | 0.27 (0.04-1.94) | 0.192 |
| MUC4 p.Gly3372Asp | 0.50 (0.21-1.16) | 0.107 | 0.37 (0.16-0.86) | 0.022 |
| MUC4 p. His2381Asp | 0.28 (0.09-0.89) | 0.031 | 0.43 (0.13-1.38) | 0.156 |
| MUC4 p. His2413Gln | 0.32 (0.08-1.31) | 0.113 | 0.23 (0.03-1.67) | 0.146 |
| MUC4 p. Pro3360His | 0.46 (0.24-0.88) | 0.020 | 0.71 (0.37-1.35) | 0.290 |
| MUC4 p.Ser3370Thr | 0.39 (0.12-1.24) | 0.110 | 0.34 (0.11-1.10) | 0.073 |
| MUC4 p. Thr2382Ala | 0.35 (0.16-0.79) | 0.011 | 0.31 (0.12-0.79) | 0.014 |
| MUC4 p. Thr2398Ala | 0.04 (0.00-2.76) | 0.138 | 0.17 (0.02-1.24) | 0.080 |
| MUC4 p. Thr2411Ser | 0.46 (0.25-0.86) | 0.015 | 0.35 (0.18-0.71) | 0.004 |
| MUC4 p. Thr3350Asn | 0.39 (0.12-1.24) | 0.110 | 0.34 (0.11-1.10) | 0.073 |
| MUC4 p. Thr3355Ser | 0.39 (0.21-0.69) | 0.002 | 0.45 (0.26-0.80) | 0.006 |
| MUC4 p. Val3353Ala | 0.48 (0.23-1.00) | 0.049 | 0.48 (0.24-0.97) | 0.040 |
| USH2A\|LOC102723833 | 0.58 (0.33-1.01) | 0.054 | 0.37 (0.20-0.67) | 0.001 |
| p.Leu1658Pro\| | | | | |

#Hazard ratio (HR [5% confidence interval (CI)] of tumor recurrence

Similar results have been found in the analysis of the survival of patients by univariant logistics regression. A total of 13 and 17 variants significantly correlated with increased risk of dead of patients detected from the tissue before and after CCRT respectively. Most variants were also within the coding region of MUC17 and MUC4 gene. MUC17 p.Thr2702Val, detected from both before and after CCRT-treated tissues, was also the predominant variant related to the survival of patients (Tissue before CCRT, OR[95% CI]=4.22 (2.20-8.11), p<0.001; Tissue after CCRT, OR[95% CI]=4.47 (2.35-8.49), p<0.001, Table 9). The variation of MUC4 p.Thr3355Ser was shown to be associated with reduced risk for death as well (before CCRT, OR[95% CI]=0.40 (0.21-0.74), p=0.004; after CCRT, OR[95% CI]=0.53 (0.29-0.97), p=0.039, Table 9). Notably, the variant MYH4 p.Gln1210fs within MYH4 gene, detected from both before and after CCRT tissue, also showed significant correlated with reduced risk for dead of patients (before CCRT, OR[95% CI]=0.38 (0.17-0.87), p=0.022; after CCRT, OR[95% CI]=0.38 (0.18-0.79), p=0.010, Table 9).

TABLE 9

| Variants | HR (95% CI)# | P-value* | HR (95% CI)# | P-value* |
|---|---|---|---|---|
| EP300 p.Glu1523Lys | 0.61 (0.24-1.54) | 0.263 | 0.42 (0.20-0.91) | 0.028 |
| MUC16 p.Thr5382Lys | 0.46 (0.14-1.49) | 0.195 | 0.44 (0.16-1.23) | 0.118 |
| MUC17 p.Asn2706Ser | 4.13 (2.14-7.97) | <0.001 | 2.76 (1.36 -. 5.58) | 0.005 |
| MUC17 p.Leu2703 | 3.86 (1.99-7.47) | <0.001 | 2.48 (1.20-5.14) | 0.014 |
| _Leu2704delinsProVal | | | | |
| MUC17 p.Leu2712Val | 4.00 (2.07-7.74) | <0.001 | 2.48 (1.20-5.14) | 0.014 |
| MUC17 p.Pro1319Ser | 2.13 (1.16-3.92) | 0.015 | 2.03 (1.10-3.75) | 0.023 |
| MUC17 p.Pro2716Ala | 3.86 (1.99-7.47) | <0.001 | 2.48 (1.20-5.14) | 0.014 |
| MUC17 p.Ser656Thr | 0.05 (0.00-370.06) | 0.505 | — | — |
| MUC17 p.Thr2702Val | 4.22 (2.20-8.11) | <0.001 | 4.47 (2.35-8.49) | <0.001 |
| MUC17 p.Thr2721Ile | 3.32 (1.68-6.57) | 0.001 | 2.43 (1.22-4.82) | 0.012 |
| MUC4 p.Ala2390Thr | 0.33 (0.08-1.38) | 0.130 | 0.25 (0.04-1.85) | 0.170 |
| MUC4 p.Asp2397His | 0.29 (0.07-1,18) | 0.084 | 0.20 (0.03-1.44) | 0.110 |
| MUC4 p.Gly3372Asp | 0.60 (0.24-1.52) | 0.280 | 0.34 (0.13-8.86) | 0.022 |
| MUC4 p. His2381Asp | 0.28 (0.07-1.15) | 0.077 | 0.30 (0.07-1.23) | 0.094 |
| MUC4 p. His2413Gln | 0.23 (0.03-1.67) | 0.146 | 0.04 (0.00-2.79) | 0.136 |
| MUC4 p. Pro3360His | 0.47 (0.23-0.96) | 0.037 | 0.65 (0.32-1.32) | 0.238 |
| MUC4 p.Ser3370Thr | 0.53 (0.17-1.73) | 0.295 | 0.23 (0.06-0.97) | 0.045 |
| MUC4 p. Thr2382Ala | 0.35 (0.14-0.89) | 0.028 | 0.13 (0.03-0.54) | 0.005 |
| MUC4 p. Thr2398Ala | 0.04 (0-6.11) | 0.214 | 0.04 (0.00-2.14) | 0.112 |
| MUC4 p. Thr2411Ser | 0.50 (0.26-0.98) | 0.042 | 0.33 (0.15-0.71) | 0.005 |
| MUC4 p. Thr3350Asn | 0.53 (0.17-1.73) | 0.295 | 0.23 (0.06-0.97) | 0.045 |
| MUC4 p. Thr3355Ser | 0.40 (0.21-0.74) | 0.004 | 0.53 (0.29-0.97) | 0.039 |
| MUC4 p. Val3353Ala | 0.34 (0.14-0.80) | 0.014 | 0.35 (0.16-0.76) | 0.008 |
| MYHAS MYH4 | 0.38 (0.17-0.87) | 0.022 | 0.38 (0.18-0.79) | 0.010 |
| Ip.Gln1210fs | | | | |
| PCLO p. Val196Phe | 0.04 (0.00-8.88) | 0.248 | 0.04 (0.00-8.88) | 0.248 |
| USH2A\|LOC102723833 | 0.57 (0.31-1.03) | 0.060 | 0.36 (0.19-0.68) | 0.002 |
| p.Leu1658Pro\| | | | | |

# Hazard ratio (HR [95% confidence interval (CI)] of death
\hat{ }Univariate Cox regression

We analyzed the collinearity of unfavorable variants for prognosis response within both the MUC17 and MUC4 genes with collinearity diagnostics. None of the variants displayed a high degree of genetic collinearity (data not shown). We defined the variant of MUC17 p.Thr2702Val and the wild-type of MUC4 p.Thr3355Ser as unfavorable genotypes. Patients carrying at least one of the unfavorable genotypes (positive) revealed 100% sensitivity to predict disease recurrence in tumor tissue before CCRT (P<0.001, Tables 10-11, Fisher's exact test).

TABLE 10

| | | No | | |
|---|---|---|---|---|
| | Unfavorable | recurrence | Recurrence | |
| Variants (before CCRT) | genotype | 11 (17.7) 51 | (82.3) | p-value* |
| \hat{ }MUC17 | Negative | 11 (45.8) | 13 (54.2) | <0.001 |
| p.Thr2702Val(v) + | | | | |
| MUC4 p. Thr3355Ser(w) | Positive | 0 (0) | 38 (100) | |

*Pearson's Chi-square test or Fisher's exact test
\hat{ }Unfavorable genotypes: MUC17 p.Thr2702Val(v, variation) and MUC4 p. Thr3355Ser(w, wild-type)

TABLE 11

| | | No | Recurrence | |
|---|---|---|---|---|
| | Unfavorable | recurrence | 51 | p- |
| Variants (After CCRT) | genotype | 11 (17.7) | (82.3) | value* |
| \hat{ }MUC17 | Negative | 11 (37.9) | 18 (62.1) | <0.001 |
| p.Thr2702Val(v) + | | | | |
| MUC4 p. Thr3355Ser(w) | Positive | 0 (0) | 33 (100) | |

*Pearson's Chi-square test or Fisher's exact test
\hat{ }Unfavorable genotypes: MUC17 p.Thr2702Val(v, variation) and MUC4 p. Thr3355Ser(w, wild-type)

Univariate Cox regression further revealed a 4.57-fold increased hazard for recurrence detected from fresh esophageal tumor tissue (HR [95% CI]=4.57 (2.31-9.01), p<0.001, Table 12; after CCRT, OR[95% CI]=3.43 (1.90-6.17), p<0.001, Table 13). Univariate Cox regression also demonstrated the evidently increased risk of up to 4.98 folds for death in fresh tissues (HR [95% CI]=4.98 (2.34-10.58), p<0.001, Table 12).

TABLE 12

| | Favorable | | |
|---|---|---|---|
| Variants (Before CCRT) | genotype | HR (95% CI)# | P-value* |
| MUC17 p.Thr2702Val(v) + | Negative | 1 | |
| MUC4 p. Thr3355Ser(w) | Positive | 4.57 (2.31-9.01) | <0.001 |

#Hazard ratio (HR [5% confidence interval (CI)] of mortality
\hat{ }Univariate Cox regression
*Unfavorable genotypes: MUC17 p.Thr2702Val(v, variation) and MUC4 p. Thr3355Ser(w, wild-type)

TABLE 13

| | Favorable | | |
|---|---|---|---|
| Variants (After CCRT) | genotype | HR (95% CI)# | P-value* |
| \hat{ }MUC17 p. Thr2702Val(v) + | Negative | 1 | |
| MUC4 p. Thr3355Ser(w) | Positive | 3.43 (1.90-6.17) | <0.001 |

# Hazard ratio (HR [5% confidence interval (CI)] of tumor recurrence
*Univariate Cox regression
\hat{ }Unfavorable genotypes: MUC17 p.Thr2702Val(v, variation) and MUC4 p. Thr3355Ser(w, wild-type)

MUC17 p.Thr2702Val and MUC4 p.Thr3355Ser can predict the risk of death for 92.1% sensitivity in tumor tissues before CCRT treatment (P<0.001, Table 14). MUC17 p.Thr2702Val and MUC4 p.Thr3355Ser can also predict the risk of death for 90.9% sensitivity in tumor tissues after CCRT treatment (P=0.003, Table 15).

TABLE 14

| | Unfavorable | Alive | Dead | p- |
|---|---|---|---|---|
| Variants (Before CCRT) | genotype | 11 (17.7) | 51 (82.3) | value* |
| \hat{ }MUC17 | Negative | 13 (54.2) | 11 (45.8) | <0.001 |
| p.Thr2702Val(v) + | | | | |
| MUC4 p. Thr3355Ser(w) | Positive | 3 (7.9) | 35 (92.1) | |

*Pearson's Chi-square test or Fisher's exact test
\hat{ }Unfavorable genotypes: MUC17 p.Thr2702Val(v, variation) and MUC4 p. Thr3355Ser(w, wild-type)

TABLE 15

| | Unfavorable | Alive | Dead | p- |
|---|---|---|---|---|
| Variants (After CCRT) | genotype | 16 (25.8) | 46 (74.2) | value* |
| \hat{ }MUC17 | Negative | 13 (44.8) | 16 (55.2) | 0.003 |
| p.Thr2702Val(v) + | | | | |
| MUC4 p. Thr3355Ser(w) | Positive | 3 (9.1) | 30 (90.9) | |

*Pearson's Chi-square test or Fisher's exact test
\hat{ }Unfavorable genotypes: MUC17 p.Thr2702Val(v, variation) and MUC4 p. Thr3355Ser(w, wild-type)

Univariate Cox regression demonstrated that the risk of death in fresh (before CCRT) or CCRT-treated tissue with unfavorable genotypes of MUC17 p.Thr2702Val and/or MUC4 p.Thr3355Ser was 4.98 fold and 3.55 folds higher than those without unfavorable genotypes, respectively (before CCRT, OR[95% CI]=4.98 (2.34-10.58), p<0.001, Table 16; after CCRT, OR[95% CI]=3.55 (1.86-6.79), p<0.001, Table 17).

TABLE 16

| | Unfavorable | | |
|---|---|---|---|
| Variants (Before CCRT) | genotype | HR (95% CI)# | P-value* |
| \hat{ }MUC17 | Negative | 1 | |
| p.Thr2702Val(v) + | | | |
| MUC4 p. Thr3355Ser(w) | Positive | 4.98 (2.34-10.58) | <0.001 |

# Hazard ratio (HR [5% confidence interval (CI)] of mortality
*Univariate Cox regression
\hat{ }Unfavorable genotypes: MUC17 p.Thr2702Val(v, variation) and MUC4 p. Thr3355Ser(w, wild-type)

TABLE 17

| | Unfavorable | | |
|---|---|---|---|
| Variants (After CCRT) | genotype | HR (95% CI)# | P-value\hat{ } |
| MUC17 | Negative | 1 | |
| p.Thr2702Val(v) + | | | |
| MUC4 p. Thr3355Ser(w) | Positive | 3.55 (1.86-6.79) | <0.001 |

# Hazard ratio (HR [5% confidence interval (CI)] of mortality
\hat{ }Univariate Cox regression
\hat{ }Unfavorable genotypes: MUC17 p.Thr2702Val(v, variation) and MUC4 p. Thr3355Ser(w,wild-type)

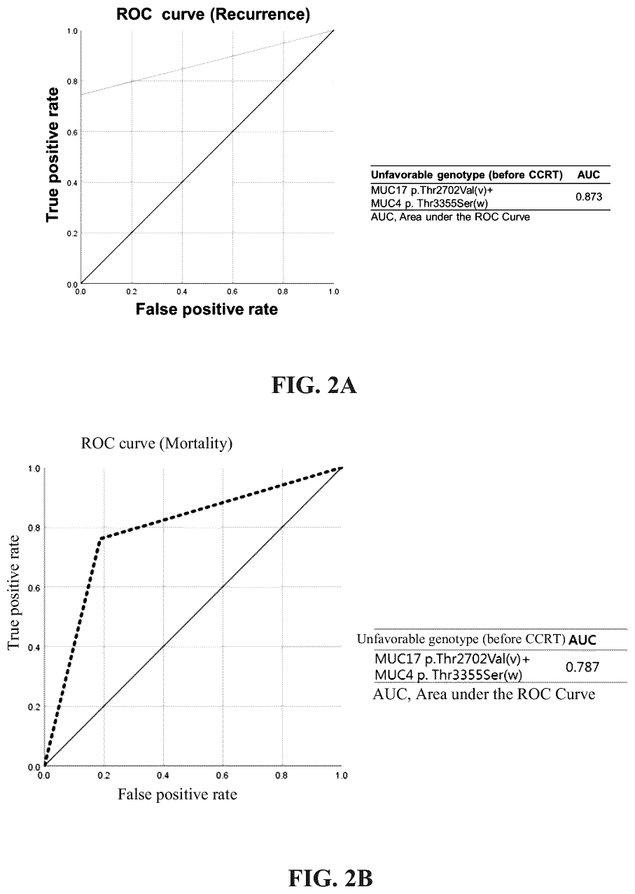

FIGS. 2A and 2B show that receiver operating characteristic (ROC) curves for risk genotypes of the tissues before CCRT were used to differentiate the ESCC patients with (2A) disease recurrence or non-recurrence and (2B) dead or alive. AUC, area under the ROC curve. ROC curve revealed that the positive of either the unfavorable genotype of MUC17 p.Thr2702Val or MUC4 p.Thr3355Ser had excellent capability for predicting tumor recurrence (AUC=0.873, FIG. 2A), and a fair discriminative ability for the mortality of patients (AUC=0.787, FIG. 2B).

Patients who were with or without carrying favorable genotypes exhibited strong significantly difference in the distribution of both progression-free (PFS) and overall survival (OS) (p<0.001 by log-rank test, respectively).

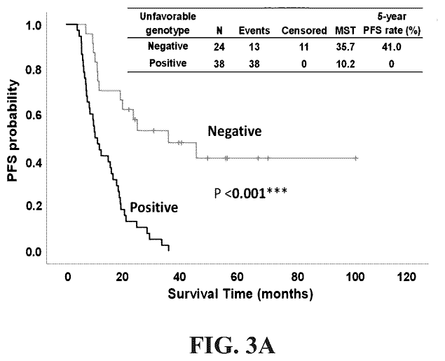

FIGS. 3A and 3B show Kaplan-Meier estimates of tumor progression-free survival (PFS, A) and overall survival (OS, B) by patients carrying unfavorable genotypes (positive) or not (negative) in tissue before CCRT. Unfavorable genotypes: MUC17 p.Thr2702Val(variant) and MUC4 p.Thr3355Ser(wild-type); MST, median survival time. The median PFS was 35.7 and 10.2 months for the negative and positive groups, respectively (FIG. 3A). Meanwhile, none of the unfavorable genotypes carriers can reach 5-year progression-free survival, in contrast to negative patients, who had a 41.0% 5-year PFS rate (FIG. 3A). For the overall survival analysis, patients without any of the unfavorable genotype can enjoy 68.4 months of median overall survival and 57.2% long-term survival. On the contrary, those carrying an unfavorable genotype only had an 18-months OS and 3.1% long-term survival (FIG. 3B).

### Example 4

### Comprehensive Comparison of Variation Changes Before and After CCRT and Analysis of Clinical Significance

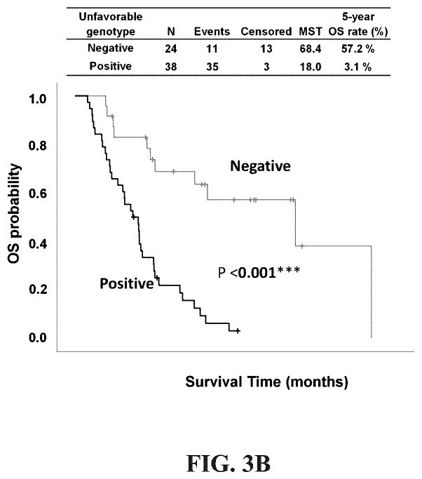

Comprehensive comparison of variation changes before and after CCRT and analysis of clinical significance is explored in this example. FIG. 4 shows an overview of tissue-site variation changes before and after CCRT, in which there are 1814 total variation sites before CCRT, and 1754 total variation sites after CCRT. The difference in variation amount between the post-CCRT and the pre-CCRT is −60.

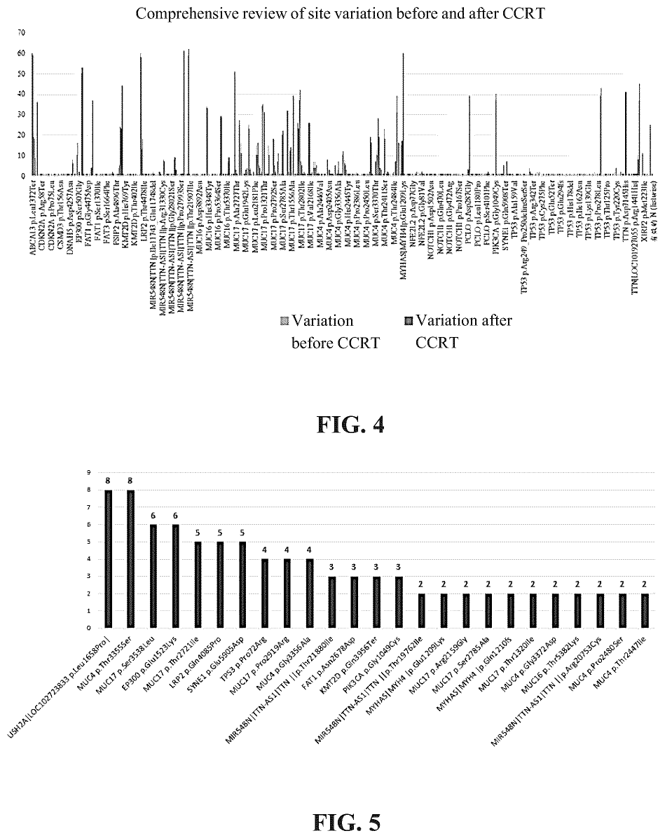

FIG. 5 shows the order of the number of increases in site variation after CCRT (≥1), that is, the total increase in site variation after CCRT is greater than 1 sample pair. The genes with the top 10 most frequently increased variants are USH2A, MUC4 (2), MUC17 (3), EP300, LRP2, SYNE1 and TP53.

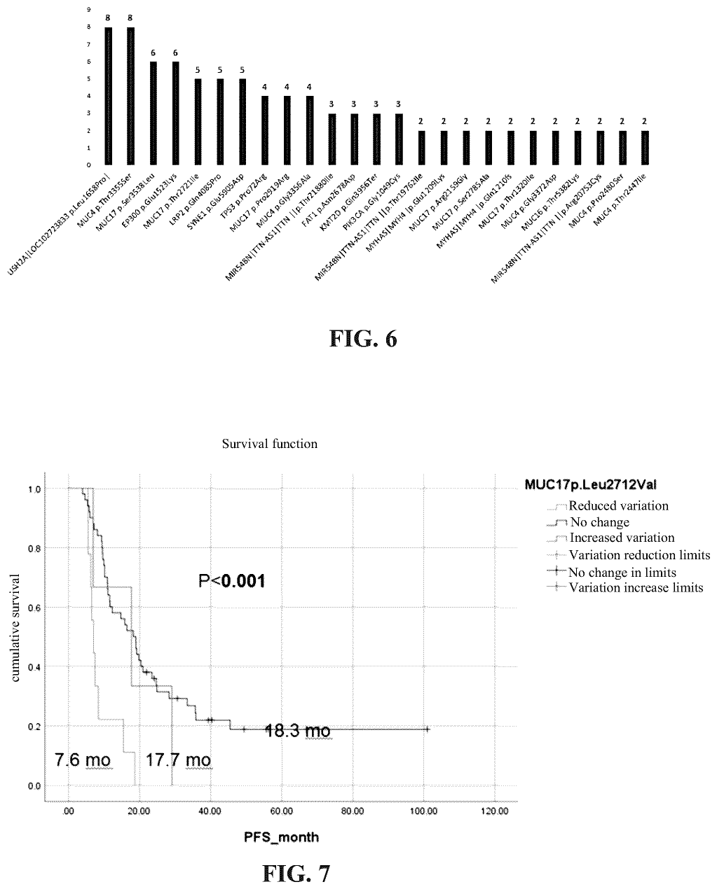

FIG. 6 shows the order of the number of sites with reduced variation after CCRT (≥1), that is, the reduction in site variation after CCRT is greater than 1 sample pair. The genes with the top 10 most frequently increased variants are MUC4 (4), MUC17 (4), ZFHX4 and TP53.

Tables 18 and 19 show the correlation analysis of site variation changes and CCRT response after CCRT, in which Table 18 is analyzed by chi-square test, and Table 19 is analyzed by univariate regression. There are three types of change types, −1 means before: there is variation; after: no variation; 0 means there is no change before and after; +1 means before: no variation; after: there is variation. The results of chi-square analysis showed that the different change types of EP300p.Glu1523Lys, SYNE1p.Glu5905Asp, MUC4p.Asp2397His and MUC4p.Ala2409Val are significantly related to CCRT treatment response (the P values are 0.019, 0.049, 0.018 and 0.049 respectively, Table 18). However, in the regression analysis, only SYNE1p.Glu5905Asp, MUC4p.Asp2397His and MUC4p.Ala2409Val showed marginally significant effects (Table 19).

TABLE 18

| | change | | pCR | | |
|---|---|---|---|---|---|
| Variation site | type# | total | 22 (35.5) | PR 40 (64.5) | p-value* |
| EP300p.Glu1523Lys | (−1) | 1 (1.6) | 1 (100.0) | 0 (0.0) | 0.019 |
| | 0 | 54 (87.1) | 16 (29.6) | 38 (70.4) | |
| | (+1) | 7 (11.1) | 5 (71.4) | 2 (28.6) | |
| SYNE1p.Glu5905Asp | (−1) | 0 (0) | 0 | 0 | 0.049 |
| | 0 | 57 (91.9) | 18 (31.6) | 39 (68.4) | |
| | (+1) | 5 (100.0) | 4 (80.0) | 1 (20.0) | |
| MUC4p.Asp2397His | (−1) | 5 (8.1) | 4 (80.0) | 1 (20.0) | 0.018 |
| | 0 | 56 (90.3) | 17 (30.4) | 39 (69.6) | |
| | (+1) | 1 (1.6) | 1 (100.0) | 0 (0) | |
| MUC4p.Ala2409Val | (−1) | 5 (8.1) | 4 (80.0) | 1 (20.0) | 0.049 |
| | 0 | 56 (90.3) | 18 (32.1) | 38 (67.9) | |
| | (+1) | 1 (1.6) | 0 (0.0) | 1 (100.0) | |
| MUC4p.Pro3360His | (−1) | 7(11.3) | 4 (57.1) | 3 (42.9) | 0.067 |
| | 0 | 51 (82.3) | 15 (29.4) | 36 (70.6) | |
| | (+1) | 4 (6.5) | 3 (75.0) | 1 (25.0) | |
| MUC4p.Ala2393 Val | (−1) | 5 (8.1) | 3 (60.0) | 2(40.0) | 0.063 |
| | 0 | 55 (88.7) | 17 (30.9) | 38 (69.1) | |
| | (+1) | 2 (3.2) | 2 (9.1) | 0 (0) | |

*Chi-square test or Fisher's exact test
#(−1), before: variation, after: no variation; (0) before and after no change; (+1) before: no variation, after: variation

TABLE 19

| | change | | | P- |
|---|---|---|---|---|
| Variation site | type# | Total | OR (95% CI)# | value\hat{ } |
| EP300p.Glu1523Lys | (−1) | 1 (1.6) | 1 | |
| | 0 | 54 (87.1) | — | — |
| | (+1) | 7 (11.1) | — | — |
| SYNE1p.Glu5905Asp | (−1) | 0 (0) | — | — |
| | 0 | 57 (91.9) | 1 | |
| | (+1) | 5 (100.0) | 0.12 (0.01-1.11) | 0.061 |
| MUC4p.Asp2397His | (−1) | 5 (8.1) | 1 | |
| | 0 | 56 (90.3) | 9.18 (0.95-88.30) | 0.055 |
| | (+1) | 1 (1.6) | — | — |
| MUC4p.Ala2409Val | (−1) | 5 (8.1) | 1 | |
| | 0 | 56 (90.3) | 8.44 (0.88-81.08) | 0.065 |
| | (+1) | 1 (1.6) | — | — |
| MUC4p.Pro3360His | (−1) | 7 (11.3) | 1 | |
| | 0 | 51 (82.3) | 3.20 (0.64-16.07) | 0.158 |
| | (+1) | 4 (6.5) | 0.44 (0.03-6.70) | 0.558 |
| MUC4p.Ala2393Val | (−1) | 5 (8.1) | 1 | |
| | 0 | 55 (88.7) | 3.35 (0.51-21.94) | 0.207 |
| | (+1) | 2 (3.2) | — | — |

\hat{ }Univariant logistic analysis
#(−1), before: variation, after: no variation; (0) before and after no change; (+1) before: no variation, after: variation

Tables 20 and 21 show the correlation analysis between site variation changes and recurrence after CCRT, in which Table 20 is analyzed by chi-square test, and Table 21 is analyzed by univariate regression. The results of chi-square analysis showed that the different change types of SYNE1p.Glu5905Asp, MUC4p.Pro3360His, MUC4p.Thr2382Ala and MUC4p.Ala2390Thr were significantly related to tumor recurrence (the P values are 0.035, 0.011, 0.030 and 0.035 respectively, Table 20). In the regression analysis, it was seen that the changes in MUC17p.Thr2721Ile and MUC4p.Pro3360His variations were significantly related to recurrence (P values are <0.001 and 0.043 respectively) (Table 21).

TABLE 20

| | | | No | | |
|---|---|---|---|---|---|
| | change | | recurrence | Recurrence | |
| Variation site | type # | total | 11 (17.7) | 51 (82.3) | p-value* |
| MUC17p.Thr2721Ile | (−1) | 5 (8.1) | 1 (20.0) | 4 (80.0) | 0.075 |
| | 0 | 47 | 6 (12.8) | 41 (87.2) | |
| | | (75.8) | | | |
| | (+1) | 10 | 4 (40.0) | 6 (60.0) | |
| | | (16.1) | | | |
| SYNE1p.Glu5905Asp | (−1) | 0 (0) | 0 | 0 | 0.035 |
| | 0 | 57 | 8 (14.0) | 49 (86.0) | |
| | | (91.9) | | | |
| | (+1) | 5 | 3 (60.0) | 2 (40.0) | |
| | | (100.0) | | | |
| MIR548NTTNASITTNp.Thr21880Ile | (−1) | 0 (0) | 0 | 0 | 0.079 |
| | 0 | 59 | 9 (15.3) | 50 (84.7) | |
| | | (95.2) | | | |
| | (+1) | 3 (4.8) | 2 (66.7) | 1 (33.3) | |
| MUC4p.Asp2397His | (−1) | 5 (8.1) | 3 (60.0) | 2 (40.0) | 0.063 |
| MUC4p.Ala2409Val | 0 | 56 | 8 (14.3) | 48 (85.7) | |
| | | (90.3) | | | |
| | (+1) | 1 (1.6) | 0 (0) | 1 (100.0) | |
| | (−1) | 5 (8.1) | 3 (60.0) | 2 (40.0) | 0.063 |
| MUC4p.Pro3360His | 0 | 56 | 8 (14.3) | 48 (85.7) | |
| | | (90.3) | | | |
| | (+1) | 1 (1.6) | 1 (0.0) | 0 (0.0) | |
| | (−1) | 7 | 4 (57.1) | 3 (42.9) | 0.011 |
| | | (11.3) | | | |
| MUC4p.Thr2382Ala | 0 | 51 | 6 (11.8) | 45 (88.2) | |
| | | (82.3) | | | |
| | (+1) | 4 (6.5) | 1 (25.0) | 3 (75.0) | |
| | (−1) | 7 | 3 (42.9) | 4 (57.1) | 0.030 |
| | | (11.3) | | | |
| MUC4p.Ala2390Thr | 0 | 51 | 6 (11.8) | 45 (88.2) | |
| | | (82.3) | | | |
| | (+1) | 4 (6.5) | 2 (50.0) | 2 (50.0) | |
| | (−1) | 4 (6.5) | 2 (50.0) | 2 (50.0) | 0.035 |
| | 0 | 57 | 8 (14.0) | 49 (86.0) | |
| | | (91.9) | | | |
| | (+1) | 1 (1.6) | 1 (100.0) | 0 (0) | |

*Chi-square test or Fisher's exact test
##, (−1), before: variation, after: no variation; (0) before and after no change; (+1) before: no variation, after: variation

TABLE 21

| | | total | | | | | | |
|---|---|---|---|---|---|---|---|---|
| | | number | | | | | p-value* | |
| | change | of | | HR | | P-value\hat{ } | (chi- | p-value# |
| Variation site | type# | changes | | (95% CI)# | | (univariate) | square) | (KM) |
| MUC17p.Leu2712Val | (−1) | 9 | (14.5) | 1 | | | 0.305 | <0.001 |
| | 0 | 50 | (80.6) | 0.23 | (0.11-0.50) | <0.001 | | |
| | (+1) | 3 | (4.8) | 0.35 | (0.09-1.31) | 0.119 | | |
| MUC17p.Thr2721Ile | (−1) | 5 | (8.1) | 1 | | | 0.075 | 0.285 |
| | 0 | 47 | (75.8) | 1.47 | (0.52-4.10) | 0.467 | | |
| | (+1) | 10 | (16.1) | 0.77 | (0.22-2.75) | 0.692 | | |
| SYNE1p.Glu5905Asp | (−1) | 0 | (0) | — | | — | 0.035 | 0.063 |
| | 0 | 57 | (91.9) | 1 | | | | |
| | (+1) | 5 | (100.0) | 0.28 | (0.07-1.17) | 0.081 | | |
| MIR548NTTNAS1TTNp.Thr21880Ile | (−1) | 0 | (0) | — | | — | 0.079 | 0.074 |
| | 0 | 59 | (95.2) | 1 | | | | |
| | (+1) | 3 | (4.8) | 0.20 | (0.03-1.43) | 0.108 | | |
| MUC4p.Asp2397His | (−1) | 5 | (8.1) | 1 | | | 0.063 | 0.154 |
| | 0 | 56 | (90.3) | 2.84 | (0.69-11.67) | 0.149 | | |
| | (+1) | 1 | (1.6) | 8.74 | (0.76-100.05) | 0.081 | | |
| MUC4p.Ala2409Val | (−1) | 5 | (8.1) | 1 | | | 0.063 | 0.200 |
| | 0 | 56 | (90.3) | 1.91 | (0.60-6.15) | 0.277 | | |
| | (+1) | 1 | (1.6) | — | | — | | |
| MUC4p.Pro3360His | (−1) | 7 | (11.3) | 1 | | | 0.011 | 0.089 |
| | 0 | 51 | (82.3) | 3.35 | (1.04-10.82) | 0.043 | | |
| | (+1) | 4 | (6.5) | 2.30 | (0.46-11.45) | 0.307 | | |
| MUC4p.Thr2382Ala | (−1) | 7 | (11.3) | 1 | | | 0.030 | 0.285 |
| | 0 | 51 | (82.3) | 1.84 | (0.66-5.13) | 0.245 | | |
| | (+1) | 4 | (6.5) | 0.83 | (0.15-4.54) | 0.831 | | |
| MUC4p.Ala2390Thr | (−1) | 4 | (6.5) | 1 | | | 0.035 | 0.259 |
| | 0 | 57 | (91.9) | 2.16 | (0.52-8.88) | 0.287 | | |

| | | | | |
|---|---|---|---|---|
| (+1) | 1 | (1.6) | — | — |

\hat{ }Univariate Cox analysis;
*Chi-square test or Fisher's exact test;
#KM survival (log-rank)
##, (−1), before: variation, after: no variation; (0) before and after no change; (+1) before: no variation, after: variation

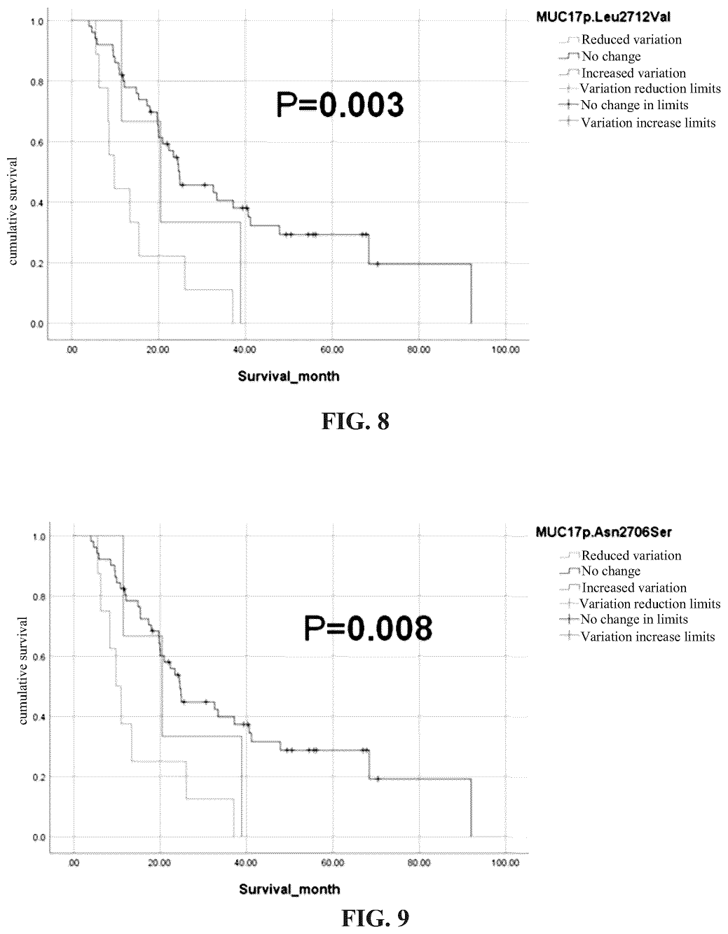

FIG. 7 shows that reduced MUC17p.Leu2712Val variation after CCRT is significantly associated with risk of shorter PFS. Variation decrease refers to tissue variation before treatment and no variation in tissue after treatment. Tables 22 and 23 show the correlation analysis between site variation changes and survival after CCRT, in which Table 22 is analyzed by chi-square test, and Table 23 is analyzed by univariate regression. The results of chi-square analysis showed that the different variation types of MUC4p.Asp2397His, MUC4p.Pro3360His and MUC4p.Ala2390Thr were significantly related to the patient's death (the P values are 0.014, 0.015, and 0.014 respectively, Table 22). In the regression analysis, it was seen that the variation of multiple sites before and after changes were significantly related to death (Table 23).

TABLE 22

| | | total | | | |
|---|---|---|---|---|---|
| | change | number | survival | death | |
| Variation site | type # | of changes | 16 (25.8) | 46 (74.2) | p-value* |
| MUC17p.Leu2712Val | (−1) | 9 (14.5) | 0 (0.0) | 9 (100.0) | 0.099 |
| MUC17p.Asn2706Ser | 0 | 50 (80.6) | 16 (32.0) | 34 (68.0) | |
| | (+1) | 3 (4.8) | 0 (0.0) | 3 (100.0) | |
| | (−1) | 8 (12.9) | 0 (0.0) | 8 (100.0) | 0.087 |
| | 0 | 51 (82.3) | 16 (31.4) | 35 (68.6) | |
| | (+1) | 3 (4.8) | 0 (0) | 3 (100.0) | |
| MUC17p.Leu2703_Leu2704delinsProVa | (−1) | 8 (12.9) | 0 (0.0) | 8 (100.0) | 0.087 |
| | 0 | 51 (82.3) | 16 (31.4) | 35 (68.6) | |
| | (+1) | 3 (4.8) | 0 (0) | 3 (100.0) | |
| MUC17p.Pro2716Ala | (−1) | 8 (12.9) | 0 (0.0) | 8 (100.0) | 0.087 |
| | 0 | 51 (82.3) | 16 (31.4) | 35 (68.6) | |
| | (+1) | 3 (4.8) | 0 (0) | 3 (100.0) | |
| MUC4p.Asp2397His | (−1) | 5 (8.1) | 4 (80.0) | 1 (20.0) | 0.014 |
| | 0 | 56 (90.3) | 12 (21.4) | 44 (78.6) | |
| | (+1) | 1 (1.6) | 0 (0) | 1 (100.0) | |
| MUC4p.Pro3360His | (−1) | 7 (11.3) | 5 (71.4) | 2 (28.6) | 0.015 |
| | 0 | 51 (82.3) | 10 (19.6) | 41 (80.4) | |
| | (+1) | 4 (6.5) | 1 (25.0) | 3 (75.0) | |
| MUC4p.Ala2390Thr | (−1) | 4 (6.5) | 3 (75.0) | 1 (25.0) | 0.014 |
| | 0 | 57 (91.9) | 12 (21.1) | 45 (78.9) | |
| | (+1) | 1 (1.6) | 1 (100.0) | 0 (0) | |

*Chi-square test or Fisher's exact test
#, (−1), before: variation, after: no variation; (0) before and after no change; (+1) before: no variation, after: variation

TABLE 23

| | | | | | | | P-value* | |
|---|---|---|---|---|---|---|---|---|
| | | | | | | | Chi- | P-value# |
| Variation site | change type & | total | | HR (95% CI)# | | P-value\hat{ } | square | KM |
| MUC17p.Leu2712Val | (−1) | 9 | (14.5) | 1 | | | 0.099 | 0.003 |
| | 0 | 50 | (80.6) | 0.29 | (0.14-0.63) | 0.002 | | |
| | (+1) | 3 | (4.8) | 0.52 | (0.14-1.95) | 0.523 | | |
| MUC17p.Asn2706Ser | (−1) | 8 | (12.9) | 1 | | | 0.087 | 0.008 |
| | 0 | 51 | (82.3) | 0.31 | (0.14-0.69) | 0.004 | | |
| | (+1) | 3 | (4.8) | 0.54 | (0.14-2.05) | 0.365 | | |
| MUC17p.Leu2703_Leu2704delinsProVa | (−1) | 8 | (12.9) | 1 | | | 0.087 | 0.006 |
| | 0 | 51 | (82.3) | 0.30 | (0.14-0.66) | 0.003 | | |
| | (+1) | 3 | (4.8) | 0.52 | (0.14-1.97) | 0.334 | | |
| MUC17p.Pro2716Ala | (−1) | 8 | (12.9) | 1 | | | 0.087 | 0.006 |
| | 0 | 51 | (82.3) | 0.30 | (0.14-0.66) | 0.003 | | |
| | (+1) | 3 | (4.8) | 0.52 | (0.14-1.97) | 0.334 | | |
| MUC4p.Asp2397His | (−1) | 5 | (8.1) | 1 | | | 0.014 | 0.128 |
| | 0 | 56 | (90.3) | 3.86 | (0.53-28.04) | 0.182 | | |
| | (+1) | 1 | (1.6) | — | | — | | |
| MUC4p.Pro3360His | (−1) | 7 | (11.3) | 1 | | | 0.015 | 0.031 |
| | 0 | 51 | (82.3) | 4.68 | (1.12-19.46) | 0.034 | | |
| | (+1) | 4 | (6.5) | 1.75 | (0.25-12.44) | 0.578 | | |
| MUC4p.Ala2390Thr | (−1) | 4 | (6.5) | 1 | | | 0.014 | 0.338 |
| | 0 | 57 | (91.9) | 2.89 | (0.40-21.04) | 0.294 | | |

| | | | | |
|---|---|---|---|---|
| (+1) | 1 | (1.6) | — | — |

\hat{ }Univariate Cox analysis;
*Chi-square test or Fisher's exact test;
#KM (Log-rank)
&, (−1), before: variation, after: no variation; (0) before and after no change; (+1) before: no variation, after: variation

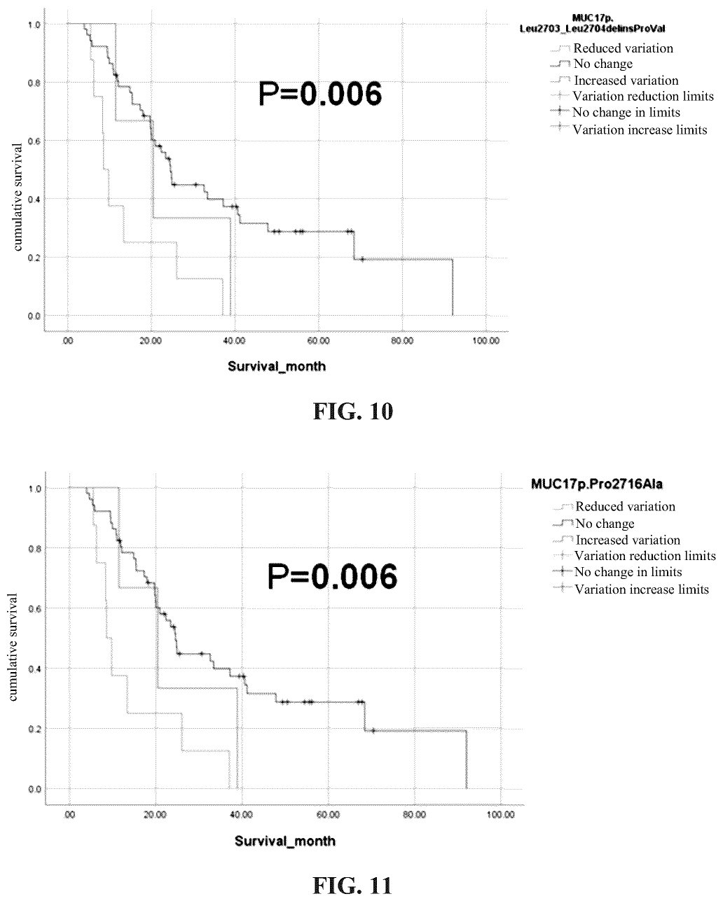

FIGS. 8-11 show that there are four MUC17 loci after CCRT, including MUC17p.Leu2712Val, MUC17p.Leu2703_Leu2704delinsProVa, MUC17p.Asn2706Ser and MUC17p.Pro2716Ala. The unchanged variation amount of the four MUC17 loci is significantly related to better overall survival (OS) time.

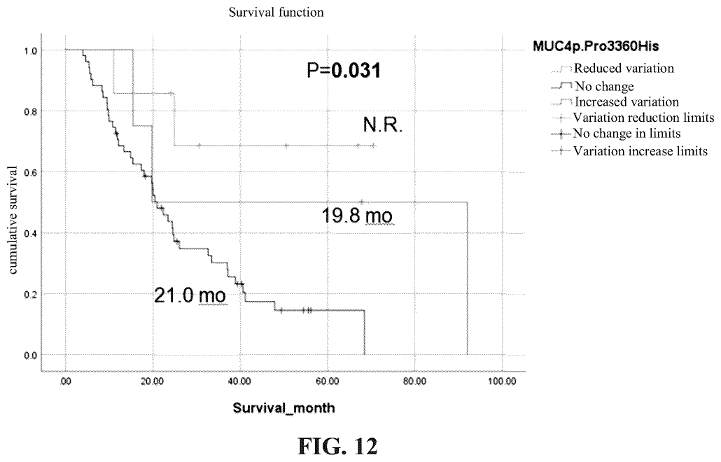

FIG. 12 shows that the reduced variation at the MUC4p.Pro3360His locus after CCRT is significantly associated with longer overall survival (OS) time. Variation decrease refers to tissue variation before treatment and no variation in tissue after treatment.

In summary, the present invention develops a next generation sequencing (NGS) kit for esophageal cancer (especially esophageal squamous cell carcinoma). Therefore, we first analyzed 402 genetic variants including 35 genes that commonly occur in esophageal squamous cell carcinoma tissue cells, and analyzed the site-specific variants in 62 pairs of esophageal squamous cell carcinoma tissues before and after CCRT to find new predictive markers. It was found that the variations at some sites would change before and after CCRT treatment, and the pattern of changes is significantly related to treatment response, recurrence and survival. The present invention combines these potential markers into an esophageal cancer detection kit, which is of extremely high value for improving the prognosis of esophageal cancer.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for evaluating treatment response, recurrence and survival by detecting genetic variants and their changes before and after concurrent chemoradiotherapy (CCRT) in tumor tissues of a patient with esophageal cancer, comprising the following steps:
(a) obtaining a tumor tissue from the patient with esophageal cancer, and extracting genomic DNA from the tumor tissue to obtain at least one genomic DNA sample; and
(b) establishing an amplicon library from the at least one genomic DNA sample and performing next generation sequencing (NGS) to obtain at least one genomic DNA datum, thereby evaluating preoperative CCRT response and prognosis in the patient with esophageal cancer;
wherein the at least one genomic DNA datum comprises a gene locus related to preoperative CCRT response and a gene locus related to prognosis in the patient with esophageal cancer, wherein when the patient with esophageal cancer having the gene locus related to preoperative CCRT response or the gene locus related to prognosis in the patient with esophageal cancer does achieve 5-year progression-free survival (PFS), indicating poor preoperative CCRT response or poor prognosis;
wherein the gene locus related to prognosis in the patient with esophageal cancer comprises Mucin 17 (MUC17) gene locus, p.Asp2397His of Mucin 4 (MUC4) gene, p.His2381Asp of MUC4 gene, p.Pro3360His of MUC4 gene, p.Thr2382Ala of MUC4 gene, p.Thr2411Ser of MUC4 gene, p.Thr3355Ser of MUC4 gene, p.Val3353Ala of MUC4 gene, USH2A gene locus, USH2A loc102723833 p.Leu1658Pro of USH2A gene and myosin, heavy chain 4 (MYH4) gene locus, p.Glu1209Lys of MYH4 gene;
wherein the MUC17 gene locus is selected from the group consisting of: p.Thr2702Val of MUC17 gene, p.Thr3355Ser of MUC17 gene, p.Leu2712Val of MUC17 gene, p.Asn2706Ser of MUC17 gene, p.Leu2703_Leu2704delinsProVal of MUC17 gene, p.Pro2716Ala of MUC17 gene, and a combination thereof.

2. The method according to claim 1, wherein the gene locus related to preoperative CCRT response is selected from the group consisting of: p.Pro1319Ser of MUC17 gene, p.Arg2159Gly of MUC17 gene, p.Gly1307Ser of MUC17 gene, p.Val1309Met of MUC17 gene, and a combination thereof.

3. The method according to claim 1, wherein the esophageal cancer is esophageal squamous cell carcinoma (ESCC).

4. The method according to claim 1, wherein the prognosis comprises recurrence and death.

5. The method according to claim 2, wherein variation of the p.Pro1319Ser of MUC17 gene in pre-treatment tissue shows that risk of partial response to preoperative CCRT in the patient with esophageal cancer is 6.22-fold than that without variation.

6. The method according to claim 1, wherein variation of the p.Thr2702Val of MUC17 gene in pre-treatment tissue shows a 3.32-fold risk of recurrence compared with that without variation.

7. The method according to claim 1, wherein variation of the p.Thr2702Val of MUC17 gene in post-treatment tissue shows a 3.21-fold risk of recurrence compared with that without variation.

8. The method according to claim 1, wherein compared with the patient with esophageal cancer after CCRT and before CCRT, situation of genetic variation comprises increase and decrease, wherein the increase refers to tissue variation after treatment and no variation before treatment, wherein the decrease refers to tissue variation before treatment and no variation after treatment.

9. The method according to claim 2, wherein variation of the combination of p.Pro1319Ser of MUC17 gene and p.Arg2159Gly of MUC17 gene in pre-treatment tissue shows that risk of partial response to preoperative CCRT in the patient with esophageal cancer is 7-fold than that without variation.

10. The method according to claim 2, wherein variation of the combination of p.Pro1319Ser of MUC17 gene and p.Gly1307Ser of MUC17 gene in pre-treatment tissue show+s that risk of partial response to preoperative CCRT in the patient with esophageal cancer is 6.03-fold than that without variation.

11. The method according to claim 2, wherein variation of the combination of p.Pro1319Ser of MUC17 gene and p.Val1309Met of MUC17 gene in pre-treatment tissue shows that risk of partial response to preoperative CCRT in the patient with esophageal cancer is 6.35-fold than that without variation.

12. A gene detection panel for evaluating treatment response, recurrence and survival by detecting genetic variants and their changes before and after concurrent chemoradiotherapy (CCRT) in tumor tissues of a patient with esophageal cancer, which is established by the method according to claim 1.

13. The gene detection panel according to claim 12, wherein the gene locus related to preoperative CCRT response is selected from the group consisting of: p.Pro1319Ser of MUC17 gene, p.Arg2159Gly of MUC17 gene, p.Gly1307Ser of MUC17 gene, p.Val1309Met of MUC17 gene, and a combination thereof.

14. The gene detection panel according to claim 12, wherein the esophageal cancer is esophageal squamous cell carcinoma (ESCC).

15. The gene detection panel according to claim 12, wherein the prognosis comprises recurrence and death.

16. The gene detection panel according to claim 13, wherein variation of the p.Pro1319Ser of MUC17 gene in pre-treatment tissue shows that risk of partial response to preoperative CCRT in the patient with esophageal cancer is 6.22-fold than that without variation.

17. The gene detection panel according to claim 12, wherein variation of the p.Thr2702Val of MUC17 gene in pre-treatment tissue shows a 3.32-fold risk of recurrence compared with that without variation.

18. The gene detection panel according to claim 12, wherein variation of the p.Thr2702Val of MUC17 gene in post-treatment tissue shows a 3.21-fold risk of recurrence compared with that without variation.

19. A method for tumor tissue evaluation of changes in gene locus variation, correlation with treatment response and prognosis in a patient with esophageal cancer before and after concurrent chemoradiotherapy (CCRT), comprising the following steps:
(a) obtaining a tumor tissue from the patient with esophageal cancer before and after CCRT treatment, and extracting genomic DNA from the tumor tissue to obtain at least one genomic DNA sample; and
(b) establishing an amplicon library from the at least one genomic DNA sample and performing next generation sequencing (NGS) to obtain at least one genomic DNA datum, thereby evaluating changes in gene locus variation in the patient with esophageal cancer before and after CCRT;
wherein the at least one genomic DNA datum comprises a gene locus related to CCRT response, wherein the changes in gene locus variation in the patient with esophageal cancer having the gene locus related to CCRT response before and after CCRT response are statistically significant;
wherein the gene locus related to CCRT response associated with treatment response is selected from the group consisting of: p.Glu1523Lys of EP300 gene, p.Glu5905Asp of SYNE1 gene, p.Asp2397His of MUC4 gene, p.Ala2409Val of MUC4 gene, p.Glu5905Asp of SYNE1 gene, p.Ala2409Val of MUC4 gene, and a combination thereof;
wherein the gene locus related to CCRT response associated with recurrence is selected from the group consisting of: p.Glu5905Asp of SYNE1 gene, p.Pro3360His of MUC4 gene, p.Thr2382Ala of MUC4 gene, p.Ala2390Thr of MUC4 gene, p.Leu2712Val of MUC17 gene, and a combination thereof;
wherein the gene locus related to CCRT response associated with survival is selected from the group consisting of: p.Asp2397His of MUC4 gene, p.Pro3360His of MUC4 gene, p.Ala2390Thr of MUC4 gene, and a combination thereof.

20. The method according to claim 19, wherein compared with the patient with esophageal cancer after CCRT and before CCRT, situation of genetic variation comprises increase and decrease, wherein the increase refers to tissue variation after treatment and no variation before treatment, wherein the decrease refers to tissue variation before treatment and no variation after treatment.
